(19) United States
(12) Patent Application Publication
(10) Pub. No.: US 2021/0403443 A1
(43) Pub. Date: Dec. 30, 2021

(54) SUBSTITUTED 1,2,4-OXADIAZOLE, ITS APPLICATION AND A PHARMACEUTICAL PREPARATION COMPRISING IT

(71) Applicant: SVENOX PHARMACEUTICALS LLC, Chicago, IL (US)

(72) Inventors: Jaroslav ROH, Jihlava (CZ); Galina KARABANOVICH, Hradec Králové (CZ); Petr PAVEK, Hradec Králové (CZ); Alexandr HRABALEK, Hradec Králové (CZ); Vera KLIMESOVA, Hradec Králové (CZ)

(21) Appl. No.: 17/293,446

(22) PCT Filed: Nov. 19, 2019
(86) PCT No.: PCT/IB2019/059934

§ 371 Date: May 12, 2021

(30) Foreign Application Priority Data
Nov. 30, 2018 (CZ) PV 2018-664

(51) Int. Cl. C07D 271/07; C07D 271/06; A61K 9/20; A61P 31/06

(52) U.S. Cl. CPC C07D 271/07; C07D 271/06; A61K 9/2009; A61K 9/2054; A61P 31/06; A61K 9/2013; A61K 9/2027; A61K 9/2018; A61K 9/2059

(57) ABSTRACT

Disclosed are compounds effective against tuberculosis based on substituted 1,2,4-oxadiazoles of general formula I, where Y═S or CH₂ and R=phenyl- or phenyl-substituted in positions 2, 3, 4, and 5 by one or several electron-acceptor groups or electron-donor groups. These compounds can be produced by easy syntheses and are characterized by low toxicity and high efficacy against mycobacteria, including multiresistant strains thereof. Also disclosed are a pharmaceutical preparation containing substituted 1,2,4-oxadiazole of formula I as an active substance as well as the use of this substituted 1,2,4-oxadiazole as an antituberculosis drug.

### FIELD OF TECHNOLOGY

The invention concerns new antituberculosis drugs based on nitro-substituted 1,2,4-oxadiazole compounds effective against sensitive as well as multiresistant mycobacterial strains.

### STATE OF THE ART

Tuberculosis (TB) is an infectious disease caused in particular by the Mycobacterium tuberculosis (Mtb.) mycobacteria that easily spread by droplet infection from persons having the pulmonary form of TB. Approximately ¼ of the world's human population is infected by the latent form of TB, of whom 5-15% are endangered by the risk of development of the active form of TB. In 2016, about 10.4 million persons contracted the disease and in 1.7 million persons TB was the cause of death (WHO—Global Tuberculosis Report 2017). These figures rank TB among the ten most frequent causes of death and in patients with AIDS, this is the most frequent cause ever. The treatment of TB includes the simultaneous administration of several drugs effective against tuberculosis for a period of 6 to 9 months, by which the side effects of the drugs, bad compliance on the part of patients, and last but not least, the expensiveness of the treatment is accentuated. Standard treatment of normal TB comprises the simultaneous administration of isoniazid, rifampicin, pyrazinamide and ethambutol for a period of 2 months of the intensive phase of treatment that is followed by 4 to 6 months of treatment by a combination of rifampicin and isoniazid. Recently, the incidence of the forms of TB resistant to first-line medicinal products (i.e. MDR-TB) is becoming increasingly common. MDR forms of TB require a special treatment that includes a cocktail of second-line medicaments, for example fluoroquinolones, amikacin, kanamycin, streptomycin, cykloserin, ethionamide, p-amino-salicylic acid, etc. Standard therapy consists five medicaments with different mechanisms of antituberculotic effect and its duration is usually 20 months. Long-term administration of such combinations of drugs may lead to the development of serious adverse events and a general decrease of patients' compliance.

For the aforementioned reasons, there is still need for finding substances that would reduce the currently employed TB therapy, be effective against MDR-TB strains as well as latent forms of TB and that would make the therapy more effective. This requires substances with a different mechanism of effect than that employed by the currently used medicaments. As far as structure is concerned, the new molecules being in pre-clinical and clinical phases of development often contain a nitro group. The nitro group seems to be of essential importance for its antimycobacterial activity. Nevertheless, such nitro-substances have different mechanisms of effect. In particular, the delamanid ((2R)-2-methyl-6-nitro-2-[(4-{4-[4-(trifluoromethoxy)phenoxy]-1-piperidinyl}phenoxy)methyl]-2,3-dihydroimidazo[2,1-b][1,3]oxazol) and pretomanid ((6S)-2-nitro-6-{[4-(trifluoromethoxy)benzyl]oxy}-6,7-dihydro-5H-imidazo[2,1-b][1,3]oxazin) nitroimidazoles, which are now in phase III of their clinical development, are concerned. Delamanid has been even approved for the treatment of the resistant forms of TB and included among recommended medicaments by the World Health Organization. (Stover, C. K.; Warrener, P.; VanDevanter, D. R.; Sherman, D. R.; Arain, T. M.; Langhorne, M. H.; Anderson, S. W.; Towell, J. A.; Yuan, Y.; McMurray, D. N.; Kreiswirth, B. N.; Barry, C. E; Baker W. R. A small-molecule nitrimidazopyran drug candidate for the treatment of tuberculosis. Nature 2000, 405, 962-966; Matsumoto, M.; Hashizume, H.; Tomishige, T.; Kawasaki, M.; Tsubouchi, H.; Sasaki, H.; Shimokawa, Y.; Komatsu, M. OPC-67683, a nitro-dihydro-imidazooxazole derivative with promising action against tuberculosis in vitro and in mice. PLOSMedicine 2006, 3, 2131-2143; Gler, M. T. et al., N. Engl. J. Med 2012, 366, 23).

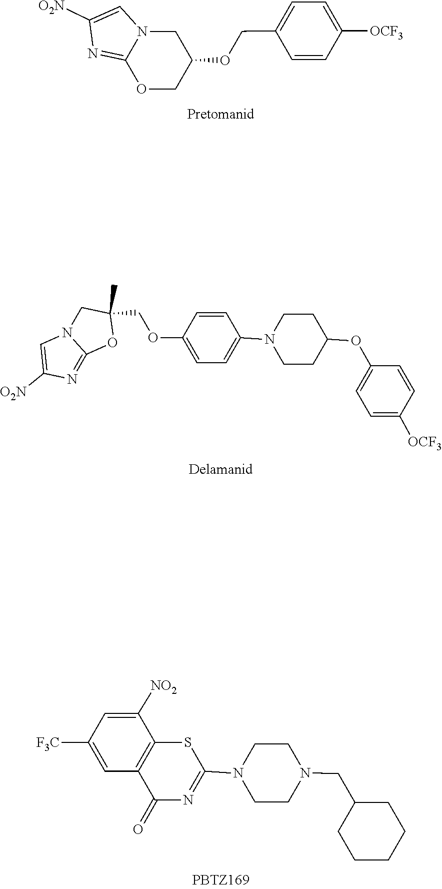 [SMILES: O=[N+]([O-])c1cn2c(n1)OC[C@@H](OCc1ccc(OC(F)(F)F)cc1)C2 ; C[C@]1(COc2ccc(N3CCC(Oc4ccc(OC(F)(F)F)cc4)CC3)cc2)Cn2cc([N+](=O)[O-])nc2O1 ; O=c1nc(N2CCN(CC3CCCCC3)CC2)sc2c([N+](=O)[O-])cc(C(F)(F)F)cc12]

The other significant group of antituberculosis drugs with effect related to the presence of a nitro group are benzothiazinones, specifically PBTZ169 (2[4-(cyclohexylmethyl)-1-piperazinyl]-8-nitro-6-(trifluoromethyl)-4H-1,3-benzothiazin-4-on) found in phase II of development (Makarov, V.; Manina, G.; Mikusova, K.; Mollmann, U.; Ryabova, O.; Saint-Joanis, B.; Dhar, N.; Pasca, M. R.; Buroni, S.; Lucarelli, A. P.; Milano, A.; De Rossi, E.; Belanova, M.; Bobovska, A.; Dianiskova, P.; Kordulakova, J.; Sala, C.; Fullnm, E.; Schneder, P.; McKinney, J. D.; Brodin, P.; Christophe, T.; Waddell, S.; Butcher, P.; Albrethesen, J.; Rosenkrands, I.; Brosch, R.; Nandi, V.; Bharath, S.; Gaonkar, S.; Shandil, R. K.; Balasubramanian, V.; Balganesh, T.; Tyagi, S.; Grosset, J.; Riccardi, G.; Cole, S .T. Benzothiazinones kill Mycobacterium tuberculosis by blocking arabinan synthesis. Science 2009, 324, 801-804).

Among other nitro compounds referred to are for example dinitrobenzamides DNB1 (N-(2-(4-methoxyphenoxy)ethyl)-3,5-dinitrobenzamid) and DNB2 (N-(2-(benzyloxy)ethyl)-3,5-dinitrobenzamid) (Christophe, T.; Jackson, M.; Jeon, H. K.; Fenistein, D.; Contreras-Dominguez, M.; Kim, J.; Genovesio, A.; Carralot, J. P.; Ewann, F.; Kim, E. H.; Lee, S. Y.; Kang, S.; Seo, M. S.; Park, E. J.; Škovierová, H.; Pham, H.; Riccardi, G.; Nam, J. Y.; Marsollier, L.; Kempf, M.; Joly-Guillou, M. L.; Oh, T.; Shin, W. K.; No, Z.; Nehrbass, U.; Brosch, R.; Cole, S. T.; Brodin, P. High content screening identifies decaprenyl-phosphoribose 2′ epimerase as a target for intracellular antimycobacterial inhibitors. PLOS Pathog 2009, 5, 1-10) or nitrofuran derivatives—for example compound Lee878 (5-(4-(4-benzylpiperazin-1-yl)phenyl)-3-(5-nitrofuran-2-yl)-4,5-dihydroisoxazol) (Hurdle J. G. et al., J. Antimicrob. Chemother. 2008, 62, 1037).

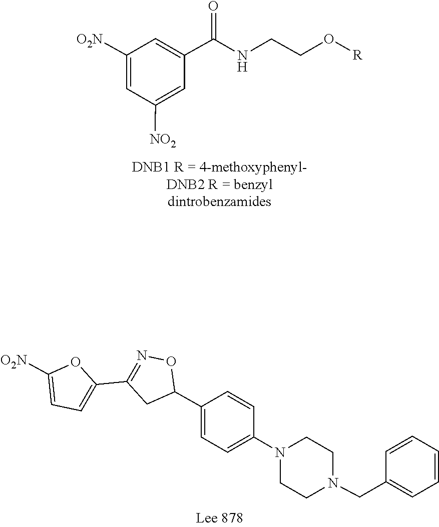

Publication FLIPO, Marion, et al. Ethionamide boosters. 2. Combining bioisosteric replacement and structure-based drug design to solve pharmacokinetic issues in a series of potent 1, 2, 4-oxadiazole EthR inhibitors. Journal of medicinal chemistry, 2011, 55.1: 68-83 discloses compounds effective and ineffective against antimycobacteria/antituberculosis. The publication deals with the relationship between the structure of the compounds and their effect. It is obvious, different structural derivatives show different or differently potent biological effects. Like many others, this publication documents that the biological activity of molecules cannot be predicted. Substituent replacement or repositioning or even a change in conformation results in an absolutely different effect of the molecule.

The closest state of the art is the WO 2014161516 document disclosing the derivatives of 1,3,4-oxadiazole and 1,3,4-thiadiazole and their effects against resistant strains of Mycobacteriom tuberculosis. This document compared to the present invention is another proof that the biological activity of molecules is absolutely inestimable in advance.

### DESCRIPTION OF THE INVENTION

The new compounds show a significant activity against Mycobacterium tuberculosis as well as atypical strains, including pathogenic and multiresistant strains isolated from ill patients. They concern namely 1,2,4-oxadiazoles with general formula I

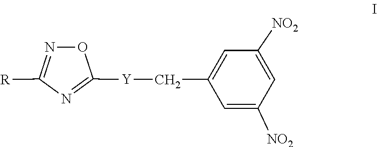

where Y═S, CH₂;
R=phenyl- or phenyl substituted in positions 2, 3, 4 and 5 by one or several electron-acceptor groups and/or one or several electron-donor groups.

Electron-donor groups refer to substituents that increase electron density on the R phenyl substituent. They concern in particular: —NH₂, —NHAl, —NAl₂, —OH, —OAl, —OAr, —NHCOCH₃, —NHCOAl; —NHCOAr; —Al, —Ar, where Alk=Alkyl, in particular the one having 1 to 4 carbon atoms, Ar=Aryl, where aryl=phenyl or phenyl substituted in positions 2, 3, 4 and 5 by one or several electron-acceptor groups and/or one or several electron-donor groups, naphthyl or pyridyl.

Electron-acceptor groups refer to substituents that decrease electron density on the R phenyl substituent. They concern in particular: —NO₂, —N⁺AlK₃, —CF₃, CCl₃, —CN, —COOH, —COOAlk, —COOAr, —CHO, —COAlk, —COAr, —F, —Cl, —Br, —I, where Alk=alkyl, in particular the one having 1 to 4 carbon atoms, Ar=Aryl, where Aryl=phenyl or phenyl substituted in positions 2, 3, 4 and 5 by one or several electron-acceptor groups and/or one or several electron-donor groups, naphthyl or pyridyl.

(source: a) John McMurry: Organic Chemistry, Sixth edition, 2004, Brooks/Cole, and Thomson Learning Company; b) L. G. Wade, Jr.: Organic Chemistry, Sixth edition, 2006, Pearson Prentice Hall Inc.; c) J. Clayden, N. Greeves, S. Warren, P. Wothers: Organic Chemistry, 2001, Oxford University Press)

Another subject of the invention is the application of the aforementioned substituted 1,2,4-oxadiazoles with general formula I according to the invention to be used as antituberculosis drug.

Another aspect of the invention is a pharmaceutical preparation containing the active substance in the form of 1,2,4-oxadiazole with formula I.

Compounds with general formula I can be obtained by commonly available procedures known in organic chemistry. During the synthesis of precursor 1,2,4-oxadiazole-5-thiols required for the synthesis of compounds with general formula I, where Y═S, known synthetic methods used in the following publications were employed: a) Xia, G.; You, X.; Liu, L.; Liu, H.; Wang, J.; Shi, Y.; Li, P.; Xiong, B.; Liu, X.; Shen, J., Design, synthesis and SAR of piperidyl-oxadiazoles as 11beta-hydroxysteroid dehydrogenase 1 inhibitors. European Journal of Medicinal Chemistry 2013, 62, 1-10; b) Burns, A. R.; Kerr, J. H.; Kerr, W. J.; Passmore, J.; Paterson, L. C.; Watson, A. J. B., Tuned methods for conjugate addition to a vinyl oxadiazole; Synthesis of pharmaceutically important motifs. Organic and Biomolecular Chemistry 2010, 8 (12), 2777-2783; c) Charton, J.; Cousaert, N.; Bochu, C.; Willand, N.; Deprez, B.; Deprez-Poulain, R. Tetrahedron Letters, 2007, vol. 48, No. 8, p. 1479-1483; d) Astellas Pharma Inc.—EP2511265, 2012, Al. (Scheme 1).

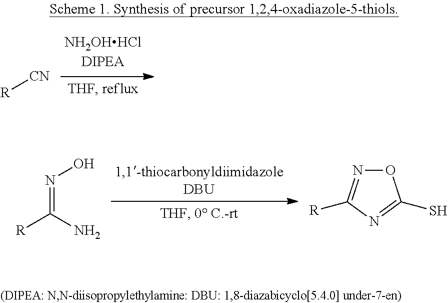

Final products with general formula I, where Y═S, were then obtained by Williamson synthesis from relevant 1,2,4-oxadiazole-5-thiol by reaction with commercially available 3,5-dinitrobenzylchloride (Scheme 2). Their preparation is not demanding in terms of synthesis and raw materials required for the preparation thereof are readily available and cheap.

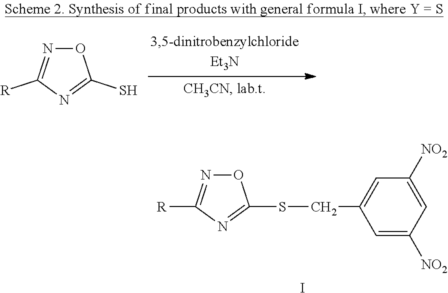

Final products with general formula I, where Y═CH₂, were prepared by methods described in references: a) Xia, G.; You, X.; Liu, L.; Liu, H.; Wang, J.; Shi, Y.; Li, P.; Xiong, B.; Liu, X.; Shen, J., Design, synthesis and SAR of piperidyl-oxadiazoles as 11beta-hydroxysteroid dehydrogenase 1 inhibitors. European Journal of Medicinal Chemistry 2013, 62, 1-10; b) Burns, A. R.; Kerr, J. H.; Kerr, W. J.; Passmore, J.; Paterson, L. C.; Watson, A. J. B., Tuned methods for conjugate addition to a vinyl oxadiazole; Synthesis of pharmaceutically important motifs. Organic and Biomolecular Chemistry 2010, 8 (12), 2777-2783.

Condensation of 3-(3,5-dinitrophenyl)propionic acid with N-hydroxy-imidamide was performed according to standard procedures known in the chemistry of peptides using water-soluble carbodiimide (El-Faham, A.; Albericio, F. Chem. Rev., 2011, 111 (11), pp 6557-6602). The by-product of the reaction (B) was then converted into the final product with general formula I by reaction with sodium methoxide in tetrahydrofuran at the ambient temperature (Scheme 3).

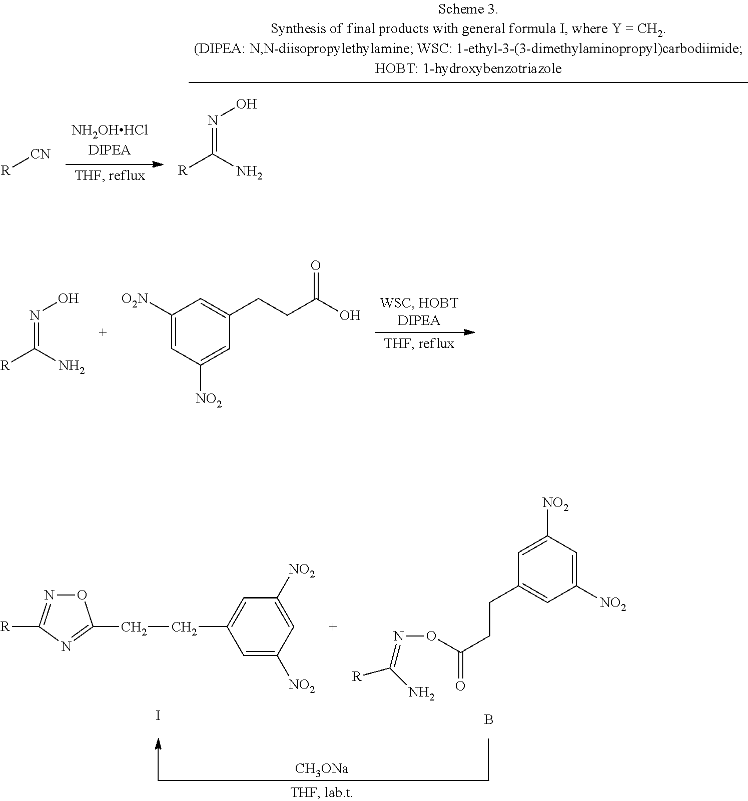

The prepared compounds with general formula I were tested by the Institute of Public Health in Ostrava (Department of Bacteriology and Mycology, Laboratory for Diagnostics of Mycobacteria, Partyzanské náměsti 7, 702 00 Ostrava) under in vitro conditions in liquid Sula's medium and their minimum inhibiting concentrations (MIC) were determined as the biological effect of molecules cannot be predicted. The antimycobacterial activity of the prepared compounds was tested on the collection strain Mycobacterium tuberculosis CNCTC My 331/88, collection atypical strains M. avium CNCTC My 330/88 and M. kansasii CNCTC My 235/80. Their activity was compared with the effect of isoniazide (INH), a commonly used medicament. The results of the tests are summarized in Table No. 3.

The most effective compounds with general formula I were further tested on multiresistant strains of mycobacteria (MDR strains) designated as Praha 1, Praha 4, Praha 131, 9449/2007, 234/2005, 7357/1998 and 8666/2010, which were clinically isolated from patients and are deposited in the Institute of Public Health in Ostrava (Department of Bacteriology and Mycology, Laboratory for Diagnostics of Mycobacteria, Partyzánské náměstí 7, 702 00 Ostrava). The values of sensitivity of these clinically isolated strains to commonly available antituberculosis drugs are summarized in Table No. 4. The antimycobacterial activities of substances with general formula I against these multiresistant strains are summarized in Table No. 5.

It has been discovered that the aforementioned substances show a highly selective antimycobacterial effect as they do not affect the viability of other types of cells. For all compounds, their cell toxicity was assessed in vitro on mammalian cell lines (the COS-1, HepG2, CHO-K1 cell lines). The respective substances do not affect the viability of mammalian cells up to the concentration of 50 μM. In addition, the respective substances do not affect the viability of standard G+ and G− bacterial strains or fungal strains up to the concentration of 250 μM.

### EXAMPLE OF INVENTION EXECUTION

Hereunder, substituted 1,2,4-oxadiazoles with general formula I will be disclosed

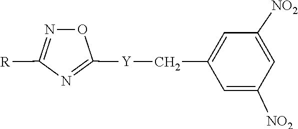

where the Y, R symbols have the meaning explained above.

### Example 1: 5-((3,5-Dinitrobenzyl)sulfanyl)-3-phenyl-1,2,4-oxadiazole (1)

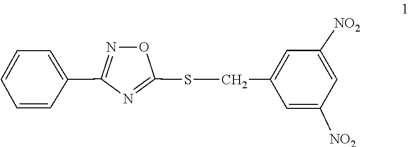 [SMILES: O=[N+]([O-])c1cc(CSc2nc(-c3ccccc3)no2)cc([N+](=O)[O-])c1]

The compound 5 -((3,5 -dinitrobenzyl)sulfanyl)-3-phenyl-1,2,4-oxadiazole 1 is prepared according to scheme 2 by reaction of 3-phenyl-1,2,4-oxadiazole-5-thiol (0.42 g, 2.34 mmol) with commercially available 3,5-dinitrobenzylchloride (97% purity, 0.47 g, 2.1 mmol) and commercially available triethylamine (0.35 ml, 2.51 mmol) in acetonitrile (30 ml) at the ambient temperature for a period of 12 hours. After the reaction was completed, acetonitrile was distilled off, the residue of evaporation was mixed with a saturated aqueous solution of NaHCO₃ (15 ml) and the solid fraction was filtered off. The acquired solid substance was resuspended in a mixture of diethylether/ethyl-acetate 2:1 (15 ml), filtered off, and the acquired crystalline 5-((3,5-dinitrobenzyl)sulfanyl)-3-phenyl-1,2,4-oxadiazole 1 was dehydrated in a dessicator. The product may be repurified by column chromatography with the use of the mobile phase hexane/ethyl-acetate 5:1.

The precursor 3-phenyl-1,2,4-oxadiazole-5-thiol was prepared by a known method (Charton, J.; Cousaert, N.; Bochu, C.; Willand, N.; Deprez, B.; Deprez-Poulain, R. Tetrahedron Letters, 2007, vol. 48, No. 8, p. 1479-1483) according to scheme 1. The other substances are generally commercially available.

### Example 2: 5-((3,5-Dinitrobenzyl)sulfanyl)-3-(p-tolyl)-1,2,4-oxadiazole (2)

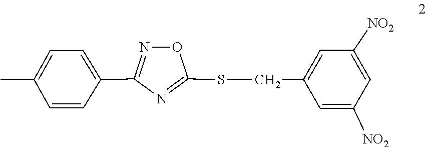 [SMILES: Cc1ccc(-c2noc(SCc3cc([N+](=O)[O-])cc([N+](=O)[O-])c3)n2)cc1]

The compound 5-((3,5-dinitrobenzyl)sulfanyl)-3-(p-tolyl)-1,2,4-oxadiazole 2 is prepared according to scheme 2 by reaction of 3-(4-methylphenyl)-1,2,4-oxadiazole-5-thiol (0.45 g, 2.34 mmol) with commercially available 3,5-dinitrobenzylchloride (97% purity, 0.47 g, 2.1 mmol) and commercially available triethylamine (0.35 ml, 2.51 mmol) in acetonitrile (30 ml) at the ambient temperature for a period of 12 hours. After the reaction was completed, acetonitrile was distilled off, the residue of evaporation was mixed with a saturated aqueous solution of NaHCO₃ (15 ml) and the solid fraction was filtered off. The acquired solid substance was resuspended in a mixture of diethylether/ethyl-acetate 2:1 (15 ml), filtered off, and the acquired crystalline 5-((3,5-dinitrobenzyl)sulfanyl)-3-(p-tolyl)-1,2,4-oxadiazol 2 was dehydrated in a dessicator. The product may be repurified by column chromatography with the use of the mobile phase hexane/ethyl-acetate 5:1.

The precursor 3-(4-methylphenyl)-1,2,4-oxadiazol-5-thiol was, according to scheme 1, the reaction of commercially available 1,8-diazabicyclo[5.4.0]undec-7-en (2 ml, 2.03 g, 0.0133 mol) with N′-hydroxy-4-methylbenzimidamide (0.5 g, 0.0033 mol) and commercially available 1.1′-thiocarbonyl-diimidazole (0.89 g, 0.005 mol) in tetrahydrofuran (20 ml) under argon atmosphere for a period of 12 hours. The solvent was then distilled off, the raw product was dissolved in water (30 ml) and washed by diethylether (1×30 ml). The aqueous layer was acidified by hydrochloric acid to pH=2 and the product was filtered off and washed by water. The acquired 3-(4-methylphenyl)-1,2,4-oxadiazole-5-thiol was recrystallized from aqueous ethanol.

The precursor N′-hydroxy-4-methylbenzimidamide was prepared by a known method invented by Murarka, Sandip; Martin-Gago, Pablo; Schultz-Fademrecht, Carsten; Al Saabi, Alaa; Baumann, Matthias; Fansa, Eyad K.; Ismail, Shehab; Nussbaumer, Peter; Wittinghofer, Alfred; Waldmann, Herbert—Chemistry—A European Journal, 2017, vol. 23, #25, p. 6083-6093. The other substances are generally commercially available.

Using the aforementioned procedures for synthesis, many other compounds with general formula I (compounds 3-10) can be synthesized.

TABLE 1
Examples of sunbstances with general formula I (compounds 1-10)

| | Y | R | Compound formula and name |
|---|---|---|---|
| 1 | S | C₆H₅ | 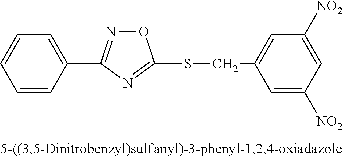 [SMILES: O=[N+]([O-])c1cc(CSc2nc(-c3ccccc3)no2)cc([N+](=O)[O-])c1] |
| 2 | S | 4-CH₃C₆H₄ | 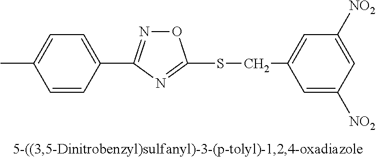 [SMILES: Cc1ccc(-c2noc(SCc3cc([N+](=O)[O-])cc([N+](=O)[O-])c3)n2)cc1] |
| 3 | S | 4-CH₃OC₆H₄ | 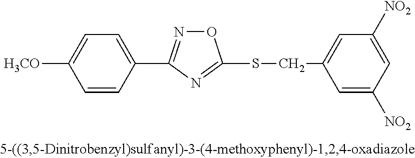 [SMILES: COc1ccc(-c2noc(SCc3cc([N+](=O)[O-])cc([N+](=O)[O-])c3)n2)cc1] |
| 4 | S | 4-ClC₆H₄ | 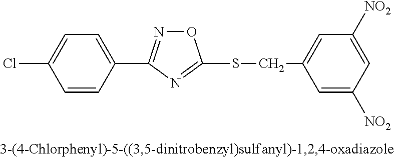 [SMILES: O=[N+]([O-])c1cc(CSc2nc(-c3ccc(Cl)cc3)no2)cc([N+](=O)[O-])c1] |
| 5 | S | 3-ClC₆H₄ | 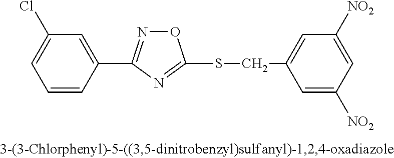 [SMILES: O=[N+]([O-])c1cc(CSc2nc(-c3cccc(Cl)c3)no2)cc([N+](=O)[O-])c1] |
| 6 | S | 2-ClC₆H₄ | 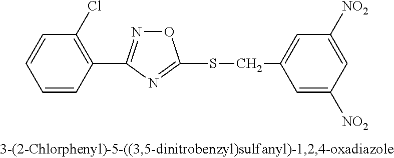 [SMILES: O=[N+]([O-])c1cc(CSc2nc(-c3ccccc3Cl)no2)cc([N+](=O)[O-])c1] |
| 7 | S | 3,4-Cl₂C₆H₃ | 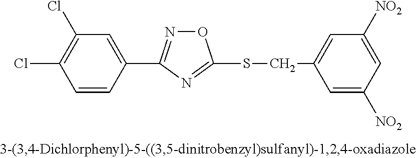 [SMILES: O=[N+]([O-])c1cc(CSc2nc(-c3ccc(Cl)c(Cl)c3)no2)cc([N+](=O)[O-])c1] |
| 8 | S | 4-FC₆H₄ | 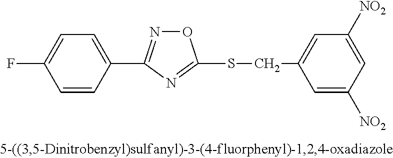 [SMILES: O=[N+]([O-])c1cc(CSc2nc(-c3ccc(F)cc3)no2)cc([N+](=O)[O-])c1] |
| 9 | S | 4-BrC₆H₄ | 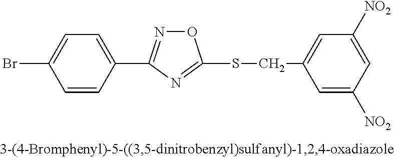 [SMILES: O=[N+]([O-])c1cc(CSc2nc(-c3ccc(Br)cc3)no2)cc([N+](=O)[O-])c1] |
| 10 | S | 4-NO₂C₆H₄ | 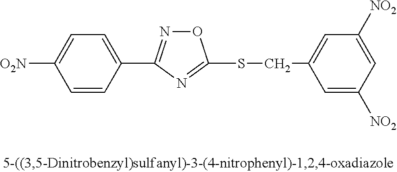 [SMILES: O=[N+]([O-])c1ccc(-c2noc(SCc3cc([N+](=O)[O-])cc([N+](=O)[O-])c3)n2)cc1] |

### Example 3: 5-(3,5-Dinitrophenethyl)-3-phenyl-1,2,4-oxadiazole (11)

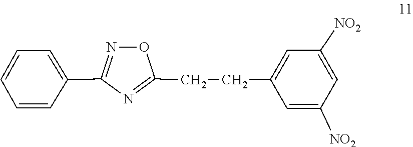 [SMILES: O=[N+]([O-])c1cc(CCc2nc(-c3ccccc3)no2)cc([N+](=O)[O-])c1]

The compound 5-(3,5-dinitrophenethyl)-3-phenyl-1,2,4-oxadiazole 11 is prepared according to scheme 3 by reaction of 3-(3,5-dinitrophenyl)propanoic acid (0.3 g, 1.25 mmol), commercially available 1-hydroxybenzotriazole hydrate (0.71 g, 4.62 mmol), commercially available diisopropylethylamine (0.65 ml, 0.48 g, 3.75 mmol), commercially available N-(3-dimethylaminopropyl)-N′-ethylcarbodiimide (0.44 ml, 0.39 g, 2.5 mmol), and N′-hydroxybenzimidamide (0.19 g, 1.38 mmol) in 30 ml of tetrahydrofuran at the boiling temperature for a period of 8 hours. After the completion of the reaction, the solvent was distilled off, the residue of evaporation was dissolved in ethyl-acetate (30 ml) and washed by saturated solution of NaHCO₃ (1×15 ml) and water (1×30 ml). The organic layer was dehydrated by anhydrous Na₂SO₄, evaporated, and the product 5-(3,5-dinitrophenethyl)-3-phenyl-1,2,4-oxadiazole 11 was separated by column chromatography (mobile phase: hexane/ethyl-acetate 5:1). The by-product, N′-((3-(3,5-dinitrophenyl)propanoyl)oxy)benzimidamide was also separated by column chromatography and can be converted into the final product 11 by reaction with 1.5 molar equivalents of sodium methanolate in tetrahydrofuran.

The precursor 3-(3,5-dinitrophenyl)propanoic acid was prepared by reduction of 3,5-dinitrocinnamic acid by the aforementioned method (Strawn, L. M.; Martell, R. E.; Simpson, R. U.; Leach, K. L.; Counsell R. E. Iodoaryl Analogues of Dioctanoylglycerol and 1-Oleoyl-2-acetylglycerol as Probes for Protein Kinase C, J. Med. Chem. 1989, 32, 2104-2110.) Hydroxylamonnium sulphate (0.96 g, 5.85 mmol) and hydroxylamin-o-sulphonic acid (2.33 g, 20.6 mmol) were added to the suspension of 3,5-dinitrocinnamic acid (0.7 g, 2.94 mmol) in 30 ml of water at 10° C. The value of pH was adjusted to 6-7 by NaOH solution. The reaction mixture was then stirred at the temperature of 10° C. for a period of 5 hours and then at the ambient temperature for a period of 12 hours. After the aforementioned time, pH was adjusted to the value of 8 by NaOH solution, the solution was filtered off and acidified to pH=2 using concentrated HCl. The aqueous solution was then extracted by ethyl-acetate (2×70 ml), the organic extract was washed by water (2×40 ml) and evaporated. 3-(3,5-Dinitrophenyl)propanoic acid was repurified by column chromatography (mobile phase: Hexane/EtOAc/CH₃COOH, 50:10:1). 3,5-Dinitrocinnamic acid was prepared from commercially available 3,5-dinitro-benzaldehyde according to the following procedure: malonic acid (0.8 g, 7.67 mmol) and 0.1 ml piperidine was added to the solution of 3,5-dinitrobenzaldehyde (1 g, 5.1 mmol) in 10 ml of pyridine. The reaction mixture was heated up to the boiling temperature of the solvent for a period of 6 hours. Then the reaction mixture was poured into 100 ml of 2M solution of HCl. The resulting precipitate was filtered off and dissolved in 70 ml of ethyl-acetate. The organic solution was washed by water (2×40 ml), dried above Na₂SO₄ and evaporated. 3,5-Dinitrocinnamic acid was repurified by column chromatography (mobile phase: Hexane/EtOAc/CH₃COOH, 40:10: 1).

The precursor N′-hydroxybenzimidamide was prepared by a known method (Burns, A. R.; Kerr, J. H.; Kerr, W. J.; Passmore, J.; Paterson, L. C.; Watson, A. J. B., Tuned methods for conjugate addition to a vinyl oxadiazole; Synthesis of pharmaceutically important motifs. Organic and Biomolecular Chemistry 2010, 8 (12), 2777-2783.).

### Example 4:5-(3,5-Dinitrophenethyl)-3-(p-tolyl)-1,2,4-oxadiazole (12)

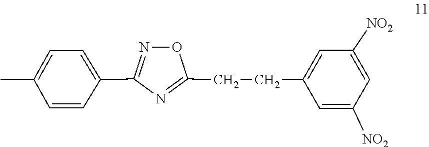 [SMILES: Cc1ccc(-c2noc(CCc3cc([N+](=O)[O-])cc([N+](=O)[O-])c3)n2)cc1]

The compound 5-(3,5-dinitrofenethyl)-3-(p-tolyl)-1,2,4-oxadiazol 12 is prepared according to scheme 3 by reaction of 3-(3,5-dinitrophenyl)propanoic acid (0.3 g, 1.25 mmol), commercially available 1-hydroxybenzotriazole hydrate (0.71 g, 4.62 mmol), commercially available diisopropylethylamine (0.65 ml, 0.48 g, 3.75 mmol), commercially available N-(3-dimethylaminopropyl)-N′-ethylcarbodiimide (0.44 ml, 0.39 g, 2.5 mmol), and N′-hydroxy-4-methylbenzimidamide (0.206 g, 1.38 mmol) in 30 ml of tetrahydrofuran at the boiling temperature of the solvent for a period of 8 hours. After the completion of the reaction, the solvent was distilled off, the residue of evaporation was dissolved in ethyl-acetate (30 ml) and washed by saturated solution of NaHCO₃ (1×15 ml) and water (1×30 ml). The organic layer was dehydrated by anhydrous Na₂SO₄, evaporated, and the product 5-(3,5-dinitrophenethyl)-3-(p-tolyl)-1,2,4-oxadiazole 12 was separated by column chromatography (mobile phase: hexane/ethyl-acetate 5:1). The by-product, N′-((3-(3,5-dinitrophenyl)propanoyl)oxy)-4-methylbenzimidamide was also separated by column chromatography and can be converted into the final product 12 by reaction with 1.5 molar equivalents of sodium methanolate in tetrahydrofuran.

The precursor 3-(3,5-dinitrophenyl)propanoic acid was prepared by reduction of 3,5-dinitrocinnamic acid by the aforementioned method (Strawn, L. M.; Martell, R. E.; Simpson, R. U.; Leach, K. L.; Counsell R. E. Iodoaryl Analogues of Dioctanoylglycerol and 1-Oleoyl-2-acetylglycerol as Probes for Protein Kinase C, J. Med. Chem. 1989, 32, 2104-2110.) Hydroxylamonnium sulphate (0.96 g, 5.85 mmol) and hydroxylamin-o-sulphonic acid (2.33 g, 20.6 mmol) were added to the suspension of 3,5-dinitrocinnamic acid (0.7 g, 2.94 mmol) in 30 ml of water at 10° C. The value of pH was adjusted to 6-7 by NaOH solution. The reaction mixture was then stirred at the temperature of 10° C. for a period of 5 hours and then at the ambient temperature for a period of 12 hours. After the aforementioned time, pH was adjusted to the value of 8 by NaOH solution, the solution was filtered off and acidified to pH=2 using concentrated HCl. The aqueous solution was then extracted by ethyl-acetate (2×70 ml), the organic extract was washed by water (2 x 40 ml) and evaporated. 3-(3,5-Dinitrophenyl)propanoic acid was repurified by column chromatography (mobile phase: Hexane/EtOAc/CH₃COOH, 50:10:1). 3,5-Dinitrocinnamic acid was prepared from commercially available 3,5-dinitrobenzaldehyde according to the following procedure: malonic acid (0.8 g, 7.67 mmol) and 0.1 ml piperidine was added to the solution of 3,5-dinitrobenzaldehyde (1 g, 5.1 mmol) in 10 ml of pyridine. The reaction mixture was heated up to the boiling temperature of the solvent for a period of 6 hours. Then the reaction mixture was poured into 100 ml of 2M solution of HCl. The resulting precipitate was filtered off and dissolved in 70 ml of ethyl-acetate. The organic solution was washed by water (2×40 ml), dried above Na₂SO₄ and evaporated. 3,5-Dinitrocinnamic acid was repurified by column chromatography (mobile phase: Hexane/EtOAc/CH₃COOH, 40:10:1).

The precursor N′-hydroxy-4-methylbenzimidamide was prepared by a known method based on the following publication: Murarka, Sandip; Martin-Gago, Pablo; Schultz-Fademrecht, Carsten; Al Saabi, Alaa; Baumann, Matthias; Fansa, Eyad K.; Ismail, Shehab; Nussbaumer, Peter; Wittinghofer, Alfred; Waldmann, Herbert—Chemistry—A European Journal, 2017, vol. 23, #25, p. 6083-6093.

Using the aforementioned procedures for synthesis, many other compounds with general formula I (compounds 13-16) can be synthesized.

TABLE 2
Examples of substances with general formula I (compounds 11-16)

| | Y | R | Compound formula and name |
|---|---|---|---|
| 11 | CH₂ | C₆H₅ | 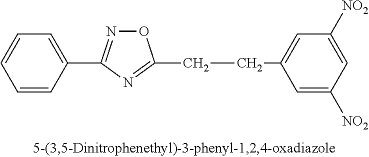 [SMILES: O=[N+]([O-])c1cc(CCc2nc(-c3ccccc3)no2)cc([N+](=O)[O-])c1] |
| 12 | CH₂ | 4-CH₃C₆H₄ | 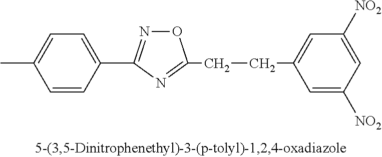 [SMILES: Cc1ccc(-c2noc(CCc3cc([N+](=O)[O-])cc([N+](=O)[O-])c3)n2)cc1] |
| 13 | CH₂ | 4-CH₃OPh | 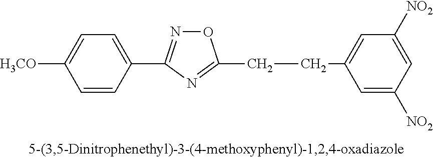 [SMILES: COc1ccc(-c2noc(CCc3cc([N+](=O)[O-])cc([N+](=O)[O-])c3)n2)cc1] |
| 14 | CH₂ | 4-ClPh | 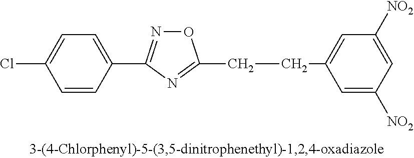 [SMILES: O=[N+]([O-])c1cc(CCc2nc(-c3ccc(Cl)cc3)no2)cc([N+](=O)[O-])c1] |
| 15 | CH₂ | 3,4-Cl₂Ph | 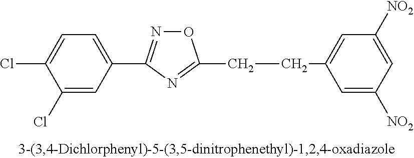 [SMILES: O=[N+]([O-])c1cc(CCc2nc(-c3ccc(Cl)c(Cl)c3)no2)cc([N+](=O)[O-])c1] |
| 16 | CH₂ | 4-FPh | 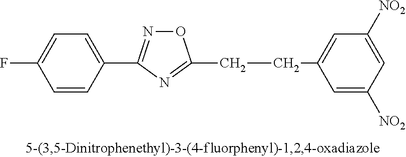 [SMILES: O=[N+]([O-])c1cc(CCc2nc(-c3ccc(F)cc3)no2)cc([N+](=O)[O-])c1] |

TABLE 3
Minimum inhibiting concentration (μmol · l⁻¹) in
vitro of substances with general formula I - the micromethod
for determination of minimum inhibiting concentrations of medicines
in Sula's medium in plastic P-plates, after 14 and 21 days of incubation
for M. tuberculosis and M. avium and after 7, 14, and
21 days of incubations for M. kansasii.

| | M. tuberculosis | M. avium | M. kansasii |
|---|---|---|---|
| | My 331/88 | My 330/88 | My 235/80 |
| 1 | 0.5/1 | 250/250 | 0.5/1/1 |
| 2 | 0.5/1 | 250/250 | 0.5/1/2 |
| 3 | 0.5/0.5 | 250/250 | 0.5/1/2 |
| 4 | 0.5/1 | 250/250 | 1/2/4 |
| 5 | 0.5/1 | 250/250 | 2/2/4 |
| 6 | 0.5/1 | 125/125 | 8/16/32 |
| 7 | 1/1 | 250/250 | 1/1/2 |
| 8 | 0.25/0.5 | 250/250 | 0.5/1/2 |
| 9 | 1/1 | 125/125 | 1/1/2 |
| 10 | 0.5/1 | 250/250 | 2/2/4 |
| 11 | 4/4 | 250/250 | 2/4/8 |
| 12 | 2/4 | 250/250 | 2/4/4 |
| 13 | 0.5/1 | 250/250 | 0.5/1/2 |
| 14 | >32/>32 | 250/250 | >32/>32/>32 |
| 15 | >32/>32 | 250/250 | 32/>32/>32 |
| 16 | >32/>32 | 250/250 | >32/>32/>32 |

TABLE 4
Minimum inhibiting concentrations (μmol · l⁻¹) in vitro of commonly
used antibiotics and antituberculosis medicaments - the micromethod for
determination of minimum inhibiting concentrations of medicines in Sula's
medium in plastic P-plates - for multiresistant strains M. tuberculosis.

| | M. tuberculosis | | | | | | |
|---|---|---|---|---|---|---|---|
| | Praha 1 | Praha 4 | Praha 131 | 9449/2007 | 234/2005 | 7357/1998 | 8666/2010 |
| Streptomycin | 13.7 R | >27.5 R | >27.5 R | >27.5 R | 27.5 R | >27.5 R | >27.5 R |
| Isoniazid | 14.6 R | 14.6 R | 14.6 R | 58.3 R | 14.6 R | 14.6 R | 29.2 R |
| Etambutol | 39.2 R | 19.6 R | 39.2 R | 9.8 C | 19.6 R | 19.6 R | 19.6 R |
| Rifampicin | >9.7 R | >9.7 R | >9.7 R | >9.7 R | >9.7 R | >9.7 R | >9.7 R |
| Ofloxacin | 1.38 C | >22.2 R | 22.2 R | 2.75 C | 0.69 C | 11.1 R | 11.1 R |
| Gentamicin | 1.05 C | 0.52 C | >8.37 R | 1.05 C | 0.26 C | 1.05 C | 2.09 C |
| Clofazimin | 0.53 R | 0.53 R | 0.26 C | 0.13 C | 0.06 C | 0.13 C | 2.11 R |
| Amikacin | 0.43 C | 0.85 C | >27.2 R | 0.43 C | 0.43 C | 0.85 C | 1.7 C |

R - strain resistant to the given antituberculosis drug
C - strain sensitive to the given antituberculosis drug

TABLE 5
Minimum inhibiting concentrations (μmol · l⁻¹) in vitro of substances
with general formulation I - the micromethod for determination of minimum
inhibiting concentrations of medicines in Sula's medium in plastic P-plates,
after 14 days of incubation - for multiresistant strains M. tuberculosis.

| | M. tuberculosis (MDR strains) | | | | | | |
|---|---|---|---|---|---|---|---|
| | Praha 1 | Praha 4 | Praha 131 | 9449/2007 | 234/2005 | 7357/1998 | 8666/2010 |
| 9 | 0.5 | 0.5 | 1 | 0.5 | 0.5 | 1 | 0.5 |
| 10 | 1 | 0.5 | 1 | 0.5 | 0.5 | 1 | 0.5 |
| 5 | 1 | 0.5 | 1 | 1 | 0.5 | 1 | 0.5 |
| 4 | 2 | 0.5 | 1 | 1 | 0.5 | 1 | 0.5 |
| 3 | 1 | 0.5 | 0.5 | 0.5 | 0.5 | 0.5 | 0.5 |
| 8 | 0.5 | 0.5 | 0.5 | 0.5 | 0.5 | 0.5 | 0.5 |

TABLE 6
Melting points and NMR spectra of substances with general formula I

| | Melting | | |
|---|---|---|---|
| | point [° C.] | ¹H NMR | ¹³C NMR |
| 1 | 135-136° C. | ¹H NMR (500 MHz, Acetone) δ | ¹³C NMR (126 MHz, Acetone) δ |
| | | 9.06-9.00 (m, 2H), 8.90-8.84 (m, | 178.26, 169.11, 149.35, 142.65, |
| | | 1H), 8.12-8.01 (m, 2H), 7.64-7.50 | 132.51, 130.89, 129.94, 128.10, |
| | | (m, 3H), 5.03-4.93 (m, 2H). | 127.07, 118.81, 35.85. |
| | | ¹H NMR (500 MHz, DMSO-d₆) δ | ¹³C NMR (126 MHz, DMSO-d₆) δ |
| | | 8.94 (d, J = 2.1 Hz, 2H), 8.73 (t, J = | 177.57, 167.90, 148.01, 141.57, |
| | | 2.1 Hz, 1H), 8.03-7.97 (m, 2H), | 132.08, 130.25, 129.41, 127.23, |
| | | 7.64-7.53 (m, 3H), 4.89 (s, 2H). | 125.66, 118.12, 34.74. |
| 2 | 150-152° C. | ¹H NMR (500 MHz, DMSO-d₆) δ | ¹³C NMR (126 MHz, DMSO-d₆) δ |
| | | 8.93 (d, J = 1.8 Hz, 2H), 8.72 (t, J = | 177.33, 167.85, 147.99, 142.12, |
| | | 1.8 Hz, 1H), 7.89 (d, J = 8.0 Hz, 2H), | 141.59, 130.25, 129.94, 127.16, |
| | | 7.36 (d, J = 8.0 Hz, 2H), 4.88 (s, | 122.86, 118.09, 34.71, 21.23. |
| | | 2H), 2.38 (s, 3H). | |
| 3 | 155-156° C. | ¹H NMR (500 MHz, DMSO-d₆) δ | ¹³C NMR (126 MHz, DMSO-d₆) δ |
| | | 8.92 (d, J = 2.2 Hz, 2H), 8.76-8.56 | 177.11, 167.63, 162.13, 148.01, |
| | | (m, 1H), 7.93 (d, J = 8.5 Hz, 2H), | 141.64, 130.21, 128.97, 118.10, |
| | | 7.09 (d, J = 8.5 Hz, 2H), 4.87 (s, | 117.87, 114.80, 55.62, 34.68. |
| | | 2H), 3.84 (s, 3H). | |
| 4 | 173-175° C. | ¹H NMR (500 MHz, DMSO-d₆) δ | ¹³C NMR (126 MHz, DMSO-d₆) δ |
| | | 8.93 (d, J = 2.2 Hz, 2H), 8.73 (t, J = | 177.87, 167.15, 148.04, 141.50, |
| | | 2.2 Hz, 1H), 8.01 (d, J = 8.5 Hz, 2H), | 136.84, 130.22, 129.60, 129.03, |
| | | 7.64 (d, J = 8.5 Hz, 2H), 4.89 (s, | 124.55, 118.14, 34.74. |
| | | 2H). | |
| 5 | 133-135° C. | ¹H NMR (500 MHz, DMSO-d₆) δ | ¹³C NMR (126 MHz, DMSO-d₆) δ |
| | | 8.93 (d, J = 2.1 Hz, 2H), 8.72 (t, J = | 178.07, 166.85, 147.98, 141.51, |
| | | 2.1 Hz, 1H), 7.97 (t, J = 1.9 Hz, 1H), | 134.19, 131.91, 131.40, 130.26, |
| | | 7.93 (dt, J = 1.1, 1.3 Hz, 1H), 7.67 | 127.62, 126.85, 125.73, 118.06, |
| | | (ddd, J = 8.1, 2.2, 1.1 Hz, 1H), 7.58 | 34.75. |
| | | (t, J = 7.9 Hz, 1H), 4.88 (s, 2H). | |
| 6 | 158-160° C. | ¹H NMR (500 MHz, DMSO-d₆) δ | ¹³C NMR (126 MHz, DMSO-d₆) δ |
| | | 8.89 (d, J = 2.2 Hz, 2H), 8.74 (t, J = | 177.24, 166.81, 148.06, 141.61, |
| | | 2.2 Hz, 1H), 7.94 (dd, J = 7.8, 1.7 | 133.03, 132.30, 131.90, 131.05, |
| | | Hz, 1H), 7.67 (dd, J = 8.1, 1.3 Hz, | 130.11, 127.78, 124.93, 118.12, |
| | | 1H), 7.61 (td, J = 7.7, 1.7 Hz, 1H), | 34.82. |
| | | 7.53 (td, J = 7.6, 1.4 Hz, 1H), 4.89 | |
| | | (s, 2H). | |
| 7 | 176-177° C. | ¹H NMR (500 MHz, DMSO-d₆) δ | ¹³C NMR (126 MHz, DMSO-d₆) δ |
| | | 8.95 (d, J = 2.1 Hz, 2H), 8.73 (t, J = | 178.61, 166.58, 148.31, 141.75, |
| | | 2.1 Hz, 1H), 8.19 (d, J = 2.0 Hz, 1H), | 135.18, 132.76, 132.18, 130.60, |
| | | 7.96 (dd, J = 8.4, 2.0 Hz, 1H), 7.85 | 129.32, 127.47, 126.49, 118.42, |
| | | (d, J = 8.4 Hz, 1H), 4.90 (s, 2H). | 35.04. |
| 8 | 150-152° C. | ¹H NMR (500 MHz, DMSO-d₆) δ | ¹³C NMR (126 MHz, DMSO-d₆) δ |
| | | 8.92 (d, J = 2.1 Hz, 2H), 8.72 (t, J = | 177.70, 167.13, 164.30 (d, J = |
| | | 2.1 Hz, 1H), 8.07-8.01 (m, 2H), | 249.9 Hz), 148.02, 141.56, 130.21, |
| | | 7.43-7.36 (m, 2H), 4.88 (s, 2H). | 129.80 (d, J = 9.1 Hz), 122.25 (d, J = |
| | | | 3.0 Hz), 118.10, 116.60 (d, J = |
| | | | 22.3 Hz), 34.73. |
| 9 | 175-176° C. | ¹H NMR (500 MHz, DMSO-d₆) δ | ¹³C NMR (126 MHz, DMSO-d₆) δ |
| | | 8.92 (d, J = 2.2 Hz, 2H), 8.72 (t, J = | 177.86, 167.25, 148.02, 141.48, |
| | | 2.1 Hz, 1H), 7.93 (d, J = 8.5 Hz, 2H), | 132.50, 130.20, 129.15, 125.70, |
| | | 7.77 (d, J = 8.4 Hz, 2H), 4.89 (s, | 124.88, 118.11, 34.74. |
| | | 2H). | |
| 10 | 184-185° C. | ¹H NMR (500 MHz, DMSO-d₆) δ | ¹³C NMR (126 MHz, DMSO-d₆) δ |
| | | 8.94 (d, J = 2.1 Hz, 2H), 8.73 (t, J = | 178.50, 166.68, 149.54, 148.07, |
| | | 2.1 Hz, 1H), 8.39 (d, J = 8.5 Hz, 2H), | 141.39, 131.48, 130.21, 128.65, |
| | | 8.26 (d, J = 8.5 Hz, 2H), 4.92 (s, | 124.57, 118.15, 34.79. |
| | | 2H). | |
| 11 | 120-122° C. | ¹H NMR (500 MHz, DMSO-d₆) δ | ¹³C NMR (126 MHz, DMSO-d₆) δ |
| | | 8.71 (d, J = 2.0 Hz, 2H), 8.71-8.69 | 179.29, 167.63, 148.13, 144.58, |
| | | (m, 1H), 8.00-7.96 (m, 2H), 7.60- | 131.73, 129.75, 129.42, 127.10, |
| | | 7.52 (m, 3H), 3.52-3.40 (m, 4H). | 126.33, 117.00, 30.64, 26.84. |
| 12 | 147-148° C. | ¹H NMR (500 MHz, DMSO-d₆) δ | ¹³C NMR (126 MHz, DMSO-d₆) δ |
| | | 8.71 (d, J = 2.1 Hz, 2H), 8.70-8.68 | 179.05, 167.57, 148.10, 144.59, |
| | | (m, 1H), 7.86 (d, J = 8.2 Hz, 2H), | 141.65, 129.93, 129.72, 127.02, |
| | | 7.35 (d, J = 8.2 Hz, 2H), 3.50-3.38 | 123.54, 116.95, 30.63, 26.82, |
| | | (m, 4H), 2.37 (s, 3H). | 21.20. |
| 13 | 128-129° C. | ¹H NMR (500 MHz, DMSO-d₆) δ | ¹³C NMR (126 MHz, DMSO-d₆) δ |
| | | 8.73-8.67 (m, 3H), 7.91 (d, J = 8.8 | 178.90, 167.32, 161.86, 148.12, |
| | | Hz, 2H), 7.09 (d, J = 8.8 Hz, 2H), | 144.61, 129.72, 128.78, 118.59, |
| | | 3.83 (s, 3H), 3.46-3.42 (m, 4H). | 116.97, 114.80, 55.56, 30.66, |
| | | | 26.80. |
| 14 | 182-183° C. | ¹H NMR (500 MHz, DMSO-d₆) δ | ¹³C NMR (126 MHz, DMSO-d₆) δ |
| | | 8.70 (s, 3H), 7.99 (d, J = 8.5 Hz, | 179.57, 166.87, 148.15, 144.53, |
| | | 2H), 7.63 (d, J = 8.5 Hz, 2H), 3.51- | 136.48, 129.73, 129.64, 128.91, |
| | | 3.42 (m, 4H). | 125.19, 117.02, 30.61, 26.84. |
| 15 | 154-155° C. | ¹H NMR (500 MHz, DMSO-d₆) δ | ¹³C NMR (126 MHz, DMSO-d₆) δ |
| | | 8.74-8.68 (m, 3H), 8.14-8.11 (m, | 179.90, 166.00, 148.13, 144.48, |
| | | 1H), 7.97-7.92 (m, 1H), 7.86-7.82 | 134.54, 132.37, 131.96, 129.80, |
| | | (m, 1H), 3.53-3.39 (m, 4H). | 128.76, 127.16, 126.82, 117.02, |
| | | | 30.53, 26.82. |
| 16 | 169-171° C. | ¹H NMR (500 MHz, DMSO-d₆) δ | ¹³C NMR (126 MHz, DMSO-d₆) δ |
| | | 8.73-8,60 (m, 3H), 8.05-7.98 (m, | 179.40, 166.84, 164.11 (d, J = |
| | | 2H), 7.42-7.31 (m, 2H), 3.51-3.40 | 249.4 Hz), 148.13, 144.55, 129.72, |
| | | (m, 4H). | 129.62 (d, J = 9.0 Hz), 122.89 (d, J = |
| | | | 3.0 Hz), 116.98, 116.59 (d, J = |
| | | | 22.1 Hz), 30.63, 26.82. |

TABLE 9
Elementary analysis of substances with general formula I

| | quantified | measured |
|---|---|---|
| 1 | C, 50.28; H, 2.81; N, 15.64; S, 8.95 | C, 50.45; H, 2.68; N, 15.52; S, 9.11 |
| 2 | C, 51.61; H, 3.25; N, 15.05; S, 8.61 | C, 51.78; H, 3.33; N, 14.96; S, 8.58 |
| 3 | C, 49.48; H, 3.11; N, 14.43; S, 8.26 | C, 49.67; H, 3.25; N, 14.24; S, 8.19 |
| 4 | C, 45.87; H, 2.31; N, 14.26; S, 8.16 | C, 45.82; H, 2.36; N, 14.18; S, 8.12 |
| 5 | C, 45.87; H, 2.31; N, 14.26; S, 8.16 | C, 45.62; H, 2.48; N, 14.61; S, 7.96 |
| 6 | C, 45.87; H, 2.31; N, 14.26; S, 8.16 | C, 45.98; H, 2.40; N, 14.37; S, 8.41 |
| 7 | C, 42.17; H, 1.89; N, 13.11; S, 7.50 | C, 42.02; H, 2.03; N, 13.0; S, 7.63 |
| 8 | C, 47.88; H, 2.41; N, 14.89; S, 8.52 | C, 44.01; H, 2.34; N, 14.97; S, 8.43 |
| 9 | C, 41.21; H, 2.07; N, 12.81; S, 7.33 | C, 41.39; H, 2.14; N, 12.72; S, 7.21 |
| 10 | C, 44.67; H, 2.25; N, 17.36; S, 7.95 | C, 44.91; H, 2.36; N, 17.27; S, 7.79 |
| 11 | C, 56.47; H, 3.55; N, 16.46 | C, 56.69; H, 3.16; N, 16.39 |
| 12 | C, 57.63; H, 3.98; N, 15.81 | C, 57.49; H, 3.7; N, 15.85 |
| 13 | C, 55.14; H, 3.81; N, 15.13 | C, 55.19; H, 3.83; N, 15.07 |
| 14 | C, 51.28; H, 2.96; N, 14.95 | C, 51.61; H, 2.84; N, 14.9 |
| 15 | C, 46.97; H, 2.46; N, 13.69 | C, 46.81; H, 2.38; N, 13.62 |
| 16 | C, 53.64; H, 3.09; N, 15.64 | C, 53.51; H, 3.03; N, 15.76 |

### Examples of Pharmaceutical Products—Tablets

In production of pharmaceutical forms, the technology usually employed in this field is opted for, i.e. dry or wet granulation, that is known to persons skilled in the art. Common and well-proven excipients and suitable agents providing the pharmaceutical form with required physical properties are used.

### Examples for Dry Granulation

### Example 1 (the content of active substance 100 mg)

| | | |
|---|---|---|
| Active substance with general formula I 1 | 100.0 | mg |
| Microcrocrystalline cellulose | 75.0 | mg |
| Sodium starch glycolate | 3.5 | mg |
| Magnesium stearate | 0.5 | mg |
| Silicone dioxide, colloidal | 0.5 | mg |

### Example 2 (the content of active substance 200 mg)

| | | |
|---|---|---|
| Active substance with general formula I 6 | 200.0 | mg |
| Microcrocrystalline cellulose | 95.0 | mg |
| Sodium starch glycolate | 7.0 | mg |
| Magnesium stearate | 1.0 | mg |
| Silicone dioxide, colloidal | 1.0 | mg |

### Example 3 (the content of active substance 300 mg)

| | | |
|---|---|---|
| Active substance with general formula I 16 | 300.0 | mg |
| Microcrocrystalline cellulose | 115.0 | mg |
| Sodium starch glycolate | 10.5 | mg |
| Magnesium stearate | 1.5 | mg |
| Silicone dioxide, colloidal | 1.5 | mg |

### Example 4 (the content of active substance 400 mg)

| | | |
|---|---|---|
| Active substance with general formula I 12 | 400.0 | mg |
| Microcrocrystalline cellulose | 130.0 | mg |
| Sodium starch glycolate | 14.5 | mg |
| Magnesium stearate | 2.0 | mg |
| Silicone dioxide, colloidal | 2.0 | mg |

### Example 5 (the content of active substance 500 mg)

| | | |
|---|---|---|
| Active substance with general formula I 9 | 500.0 | mg |
| Microcrocrystalline cellulose | 140.0 | mg |
| Sodium starch glycolate | 17.5 | mg |
| Magnesium stearate | 2.5 | mg |
| Silicone dioxide, colloidal | 2.5 | mg |

Active substance is mixed with individual ingredients of the tablet material and the mixture is transformed into tables on a tablet making machine in a customary method.

### Examples for Wet Granulation

### Example 6 (the content of active substance 100 mg)

| | | |
|---|---|---|
| Active substance with general formula I 13 | 100.0 | mg |
| Potato starch | 48.0 | mg |
| Lactose | 27.0 | mg |
| Polyvinylpyrrolidon | 3.0 | mg |
| Sodium starch glycolate | 4.0 | mg |
| Magnesium stearate | 0.2 | mg |
| Talc | 1.8 | mg |

### Example 7 (the content of active substance 200 mg)

| | | |
|---|---|---|
| Active substance with general formula I 4 | 200.0 | mg |
| Potato starch | 60.8 | mg |
| Lactose | 34.2 | mg |
| Polyvinylpyrrolidon | 6.0 | mg |
| Sodium starch glycolate | 8.0 | mg |
| Magnesium stearate | 0.4 | mg |
| Talc | 3.6 | mg |

### Example 8 (the content of active substance 300 mg)

| | | |
|---|---|---|
| Active substance with general formula I 11 | 300.0 | mg |
| Potato starch | 73.6 | mg |
| Lactose | 41.4 | mg |
| Polyvinylpyrrolidon | 9.0 | mg |
| Sodium starch glycolate | 12.0 | mg |
| Magnesium stearate | 0.6 | mg |
| Talc | 5.4 | mg |

### Example 9 (the content of active substance 400 mg)

| | | |
|---|---|---|
| Active substance with general formula I 2 | 400.0 | mg |
| Potato starch | 82.3 | mg |
| Lactose | 46.8 | mg |
| Polyvinylpyrrolidon | 12.0 | mg |
| Sodium starch glycolate | 16.0 | mg |
| Magnesium stearate | 0.8 | mg |
| Talc | 7.2 | mg |

### Example 10 (the content of active substance 500 mg)

| | | |
|---|---|---|
| Active substance with general formula I 1 | 500.0 | mg |
| Potato starch | 96.0 | mg |
| Lactose | 54.0 | mg |
| Polyvinylpyrrolidon | 15.0 | mg |
| Sodium starch glycolate | 20.0 | mg |
| Magnesium stearate | 1.0 | mg |
| Talc | 9.0 | mg |

Active substance is gradually mixed with lactose, potato starch, the mixture is granulated by polyvinylpyrrolidon, the dried granulate is mixed with sodium starch glycolate, magnesium stearate and talc and the resulting mixture is formed into tablets in a tablet making machine in a usual method.

### APPLICABILITY IN INDUSTRY

New antituberculosis drugs based on nitro-substituted 1,2,4-oxadiazole compounds effective against sensitive as well as multiresistant mycobacterial strains.

1. A substituted 1,2,4-oxadiazole with general formula I 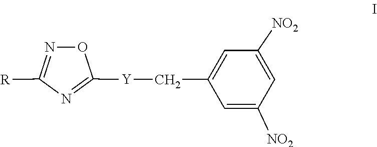
where Y═S, CH₂;
R is selected from the group comprising: phenyl- or phenyl-substituted in positions 2, 3, 4, and 5 by one or several electron-acceptor groups including —NO₂, —N+(C₁-C₄ alkyl)₃, —CF₃, CCl₃, —CN, —COOH, —COO(C₁-C₄ alkyl), —COOAryl, —CHO, —CO(C₁-C₄ alkyl), —COAryl, —F, —Cl, —Br, or —I, and/or by one or several electron-donor groups including —NH₂, —NH(C₁-C₄ alkyl), —N(C₁-C₄ alkyl)₂, —OH, —O—(C₁-C₄ alkyl), —Oaryl, —NHCOCH₃, —NHCO—(C₁-C₄ alkyl); —NHCOaryl, or —(C₁-C₄ alkyl).

2. The substituted 1,2,4-oxadiazole of general formula I according to claim 1, where Y is S.

3. The substituted 1,2,4-oxadiazole of general formula I according to claim 1, where Y is CH₂.

4. (canceled)

5. (canceled)

6. A pharmaceutical composition useful for treating tuberculosis, comprising a substituted 1,2,4-oxadiazole with general formula I 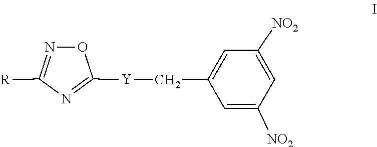
where Y═S, CH₂;
R is selected from the group comprising: phenyl- or phenyl-substituted in positions 2, 3, 4, and 5 by one or several electron-acceptor groups including —NO₂, —N+(C₁-C₄ alkyl)₃, —CF₃, CCl₃, —CN, —COOH, —COO(C₁-C₄ alkyl), —COOAryl, —CHO, —CO(C₁-C₄ alkyl), —COAryl, —F, —Cl, —Br, or —I, and/or by one or several electron-donor groups including —NH₂, —NH(C₁-C₄ alkyl), —N(C₁-C₄ alkyl)₂, —OH, —O—(C₁-C₄ alkyl), —Oaryl, —NHCOCH₃, —NHCO—(C₁-C₄ alkyl); —NHCOaryl, or —(C₁-C₄ alkyl), in a pharmaceutically acceptable carrier therefor.

7. The composition of claim 6, wherein in the substituted 1,2,4-oxadiazole of general formula I, Y is S.

8. The composition of claim 6, wherein in the substituted 1,2,4-oxadiazole of general formula I, Y is CH₂.

9. The composition of claim 6, wherein the pharmaceutically acceptable carrier comprises a dry carrier.

10. The composition of claim 6, wherein the pharmaceutically acceptable carrier comprises a wet carrier.

11. A method for treatment of tuberculosis in a patient in need of said treatment, comprising administering to said patient, a therapeutically effective amount of a substituted 1,2,4-oxadiazole with general formula I 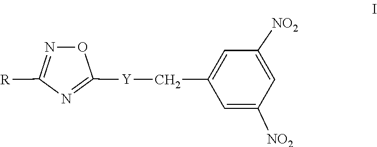
where Y═S, CH₂;
R is selected from the group comprising: phenyl- or phenyl-substituted in positions 2, 3, 4, and 5 by one or several electron-acceptor groups including —NO₂, —N+(C₁-C₄ alkyl)₃, —CF₃, CCl₃, —CN, —COOH, —COO(C₁-C₄ alkyl), —COOAryl, —CHO, —CO(C₁-C₄ alkyl), —COAryl, —F, —Cl, —Br, or —I, and/or by one or several electron-donor groups including —NH₂, —NH(C₁-C₄ alkyl), —N(C₁-C₄ alkyl)₂, —OH, —O—(C₁-C₄ alkyl), —Oaryl, —NHCOCH₃, —NHCO—(C₁-C₄ alkyl); —NHCOaryl, or —(C₁-C₄ alkyl).

12. The method of claim 11, wherein in the substituted 1,2,4-oxadiazole of general formula I, Y is S.

13. The method of claim 11, wherein in the substituted 1,2,4-oxadiazole of general formula I, Y is CH₂.

14. The method of claim 11, wherein the patient is a human.
